(19) United States
(12) Patent Application Publication
(10) Pub. No.: US 2010/0298757 A1
(43) Pub. Date: Nov. 25, 2010

(54) ADJUVANT ENHANCED ABLATION

(75) Inventor: John R. Frigstad, St. Anthony, MN (US)

Correspondence Address: WESTMAN CHAMPLIN & KELLY, P.A., SUITE 1400, 900 SECOND AVENUE SOUTH, MINNEAPOLIS, MN, 55402, US

(73) Assignee: AMS Research Corporation, Minnetonka, MN (US)

(21) Appl. No.: 12/468,668

(22) Filed: May 19, 2009

(51) Int. Cl. A61N 1/30; A61M 31/00; A61M 29/00; B65D 69/00

(52) U.S. Cl. 604/20; 604/103.02; 604/98.01; 206/570

(57) ABSTRACT

An adjuvant applicator comprises a balloon coupled to a distal end of a tube. The balloon has an inflated state and a deflated state. The exterior surface of the balloon includes a coating of an adjuvant. In one embodiment, the adjuvant is configured to absorb energy from laser light. In a method, the adjuvant applicator is provided. The balloon is inserted into the patient adjacent target tissue. The coating of adjuvant is then applied to the target tissue using the adjuvant applicator by inflating the balloon to the inflated state, which places the adjuvant in contact with the target tissue. Next, the balloon is removed from the patient and the adjuvant coated tissue is exposed to laser light. In one embodiment, the exposure of the adjuvant coated tissue to the laser light ablates the target tissue.

### CROSS-REFERENCE TO RELATED APPLICATION

The present application is based on and claims the benefit of U.S. provisional patent application Ser. No. 61/054,024, filed May 19, 2008, the content of which is hereby incorporated by reference in its entirety.

### BACKGROUND

The present invention generally relates to the application of an adjuvant to internal tissue of a patient for the purpose of enhancing an ablation treatment.

Pelvic conditions include diseases of the uterus, such as uterine fibroids and menorrhagia. Uterine fibroids are non-cancerous tumors of the uterus that that typically appear on the endometrium layer (i.e., uterine wall) of the uterus. Menorrhagia is a medical condition involving excessive and difficult to control bleeding of the endometrial layer of the uterus. These conditions have been treating through hysterectomy. However, alternative, less radical approaches are also being used.

One alternative to a hysterectomy, is endometrial ablation, which induces necrosis of the endometrial layer and a portion of the myometrial layer. These treatments can include freezing and heating the endometrial layer, or cauterizing the endometrial layer using a laser.

### SUMMARY

Embodiments of the present invention are generally directed to an adjuvant applicator configured to apply an adjuvant to internal tissue of a patient for the purpose of enhancing an ablation treatment. Specific embodiments of the invention are directed to an adjuvant applicator, a method of using the adjuvant applicator and a kit containing the adjuvant applicator.

One embodiment of the adjuvant applicator comprises a balloon coupled to a distal end of a tube. The balloon has an inflated state and a deflated state. The exterior surface of the balloon includes a coating of an adjuvant. In one embodiment, the adjuvant is configured to enhance thermal damage to the tissue.

In one embodiment of the method, an adjuvant applicator is provided comprising a balloon coupled to a distal end of a tube. The balloon has an inflated state and a deflated state. The exterior surface of the balloon includes a coating of an adjuvant. The balloon is inserted into the patient while in the deflated stated adjacent target tissue of the patient. The coating of adjuvant is then applied to the target tissue using the adjuvant applicator by inflating the balloon to the inflated state, which places the adjuvant in contact with the target tissue. The balloon is then removed from the patient. Next, the adjuvant coated target tissue is exposed to laser light. In one embodiment, the exposure of the adjuvant coated tissue to the laser light ablates the target tissue- and treats the condition of the uterus.

One embodiment of the kit comprises an adjuvant applicator, an adjuvant and a package containing the adjuvant applicator and the adjuvant. The adjuvant applicator includes a tube and a balloon coupled to a distal end of the tube. The balloon has an inflated state and a deflated state. The adjuvant is configured to absorb energy from laser light.

### BRIEF DESCRIPTION OF THE DRAWINGS

FIG. 1 is a simplified diagram of an exemplary surgical laser system.

FIG. 2 illustrates a portion of the uterine wall 114 of a female patient.

FIGS. 3A and 3B respectively illustrate an adjuvant applicator in deflated and inflated states, in accordance with embodiments of the invention.

FIG. 4 is a partial sectional view of a balloon of an adjuvant applicator in accordance with an embodiment of the invention.

FIG. 5 is a flowchart illustrating a method of applying an adjuvant to the uterine wall of a patient in accordance with embodiments of the invention.

FIG. 6 illustrates the insertion of a balloon of an adjuvant applicator into the uterus of the patient.

FIG. 7 illustrates the inflation of the balloon of an adjuvant applicator within the uterus of the patient.

FIG. 8 is a magnified view of the region of FIG. 7 within circle 8.

FIG. 9 is a kit in accordance with embodiments of the invention.

FIG. 10 is a flowchart illustrating a method of applying an adjuvant to target tissue of a patient in accordance with embodiments of the invention.

FIGS. 11A-C are simplified drawings illustrating steps of the method of FIG. 10 in accordance with embodiments of the invention.

### DETAILED DESCRIPTION OF ILLUSTRATIVE EMBODIMENTS

Embodiments of the present invention are directed to methods and devices for applying an adjuvant to target tissue of a patient for the purpose of enhancing laser tissue ablation. In general, the adjuvant is selected such that it will absorb the wavelength of the laser light to be applied to the target tissue and thereby increase the energy transfer to the target tissue. Accordingly, the adjuvant coated tissue will ablate at a faster rate without increasing the power of the laser.

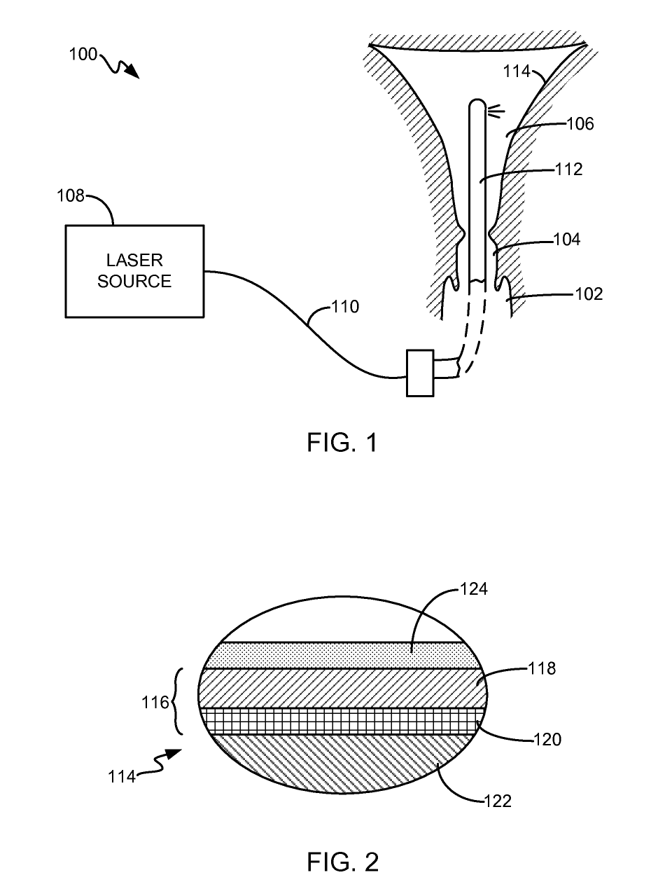

FIG. 1 is a simplified diagram of an exemplary surgical laser system 100 that is useful in the practice of embodiments of the invention. Also shown in FIG. 1 is a partial cross-sectional view of a female patient depicting the vagina 102, the cervix 104 and the uterus 106. The surgical laser system 100 comprises a laser source 108 that is configured to emit laser light. The laser light is delivered through a waveguide 110, such as an optical fiber to a probe 112. The probe 112 is configured to direct the laser light to the uterine wall 114, in accordance with conventional techniques.

The laser source 108 can produce laser light having a desired wavelength for performing surgical procedures, such as tissue ablation. In one embodiment, the laser source 108 is configured to produce an Nd:YAG operating at approximately 532 nm or 1064 nm wavelengths. The laser source 108 may be a solid state laser based on a potassium-titanyl-phosphate (KTP) crystal, a lithium triborate (LBO) laser, a beta barium borate (BBO), a holmium laser and a thulium laser, or other type of laser source used to perform tissue ablation.

The specific application of laser light to the uterine wall 114 is merely exemplary. It is understood that the surgical laser system 100 can be used to apply laser light to other tissue of the human body, such as, for example, the uterus, the colon, the prostate, the vagina, the cervix, the bladder and the anus.

FIG. 2 is a simplified cross-sectional view of a portion of the uterine wall 114 of a female patient, which comprises an endometrial layer 116 comprising a lamina fuctionalis layer 118 and a lamina basalis layer 120. Beneath the lamina basalis layer 120 is the myometrium 122. FIG. 2 also illustrates the application of an adjuvant 124 on the uterine wall 114 for absorption by the lamina fuctionalis layer 118 and the lamina basalis layer 120. Embodiments of the invention also include the application of the adjuvant 124 to other tissues of the uterus, such as fibroids.

The adjuvant 124 is biocompatible and is complimentary to the laser light emitted by the laser source 108. In one embodiment, the adjuvant 124 comprises a colorant that enhances the ablation properties of the laser light by absorbing the wavelength of the selected laser light. For example, when a KTP laser producing green laser light is used in the methods described herein, a red biocompatible colorant for the target tissue is preferably used. Examples of red biocompatible colorants that can be used with a KTP laser include Rhodamine 6G, carmine, Allura Red AC, Alizarin Red S and others. Biocompatible colorants can be obtained from chemical suppliers such Sigma-Aldrich and PolySciences, Inc., as described in U.S. Application Publication No. 2008/0039828 A1, which is incorporated herein by reference in its entirety. In one embodiment, the adjuvant 124 is suspended in a biocompatible gel matrix.

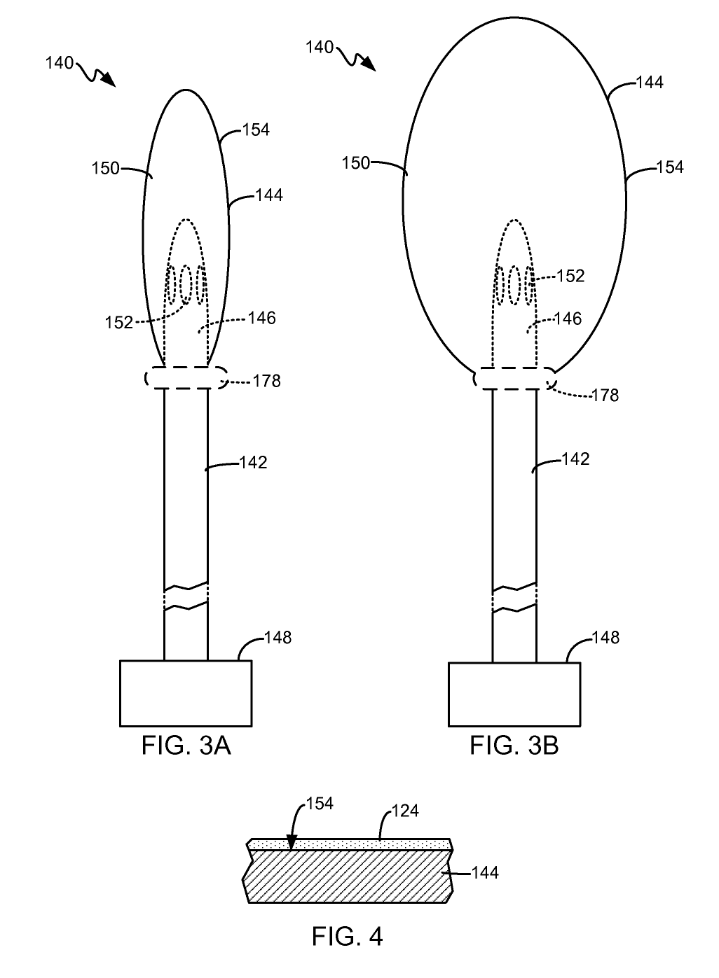

FIGS. 3A and 3B respectively illustrate an adjuvant applicator 140 in deflated and inflated states, in accordance with embodiments of the invention. In one embodiment, the applicator 140 comprises a tube 142 and a balloon 144. The balloon 144 is attached to a distal end 146 of the tube 142. A proximal end of the tube 142 is coupled to an inflator 148.

The inflator 148 operates to increase the pressure of the interior cavity 150 of the balloon 144 to transition the balloon 144 from the deflated state (FIG. 3A) to the inflated state (FIG. 3B). The inflator 148 can deliver a medium, such as liquid or gas, through the tube 148 and inject the liquid or gas into the interior cavity 150 of the balloon 144 through openings 152 to inflate the balloon 144. Embodiments of the inflator 148 include a compressor, a syringe, a pump, and other conventional components that are suitable for inflating the balloon 144. In one embodiment, the inflator 148 can release the pressure within the inflated balloon 144.

In one embodiment, the exterior surface 154 of the balloon 144 is coated with the adjuvant 124, as illustrated in the partial sectional view of the balloon 144 provided in FIG. 4. The suspension of the adjuvant 124 in a gel matrix or other viscous substance assists in adjuvant 124 adhering to the balloon 144 and uterine wall 114 of the patient. The coating of adjuvant 124 on the exterior surface 154 of the balloon 144 can be applied by the manufacturer of the applicator 140, or applied to the exterior surface 154 just prior to its insertion into the patient.

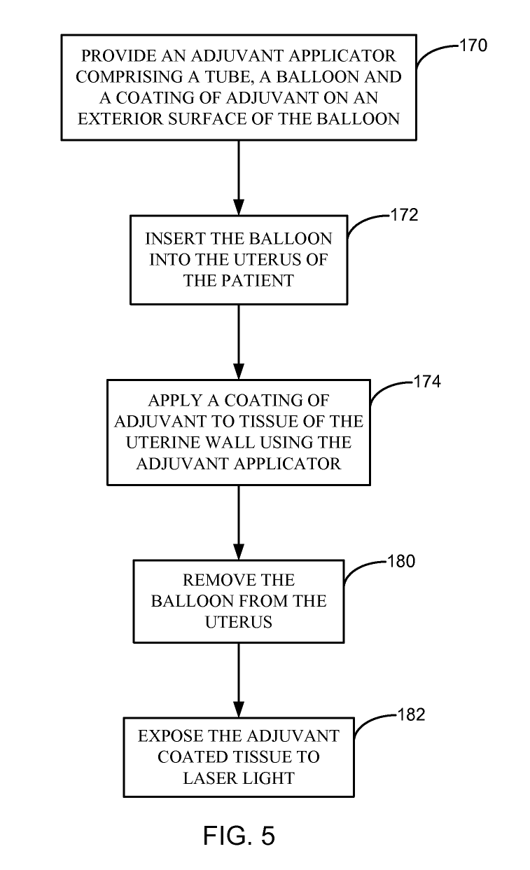

FIG. 5 is a flowchart illustrating a method of applying the adjuvant 124 to the uterine wall 114 of a patient in accordance with embodiments of the invention. At 170, an adjuvant applicator in accordance with the embodiments described above is provided. In one embodiment, the adjuvant applicator 140 comprises the tube 142, the balloon 144 and the adjuvant 124 on the exterior surface 154 of the balloon 144.

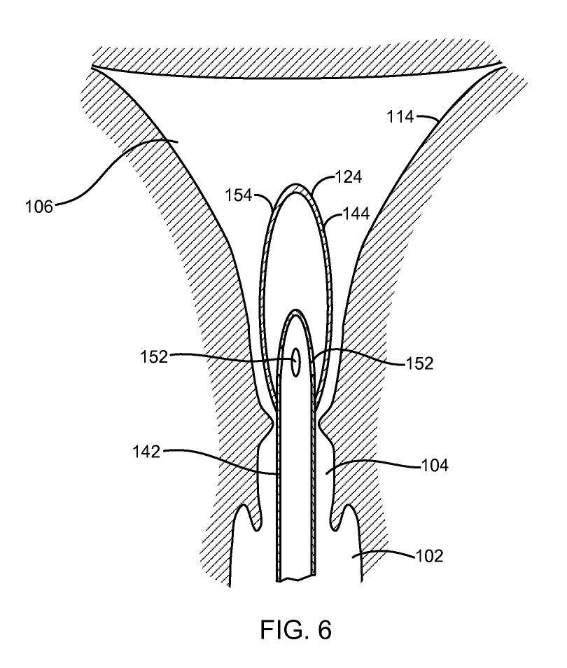

At 172. the balloon 144 is inserted into the uterus 106 of the patient while in the deflated state, as shown in FIG. 6. In one embodiment, the distal end 146 of the adjuvant applicator 140 that includes the balloon 144 is delivered to the uterus 106 in an introducer sheath, which is then removed. This prevents the vagina 102 or the cervix 104 from being exposed to the adjuvant 124. In one embodiment, the adjuvant applicator 140 is in the deflated state, as illustrated in FIG. 5.

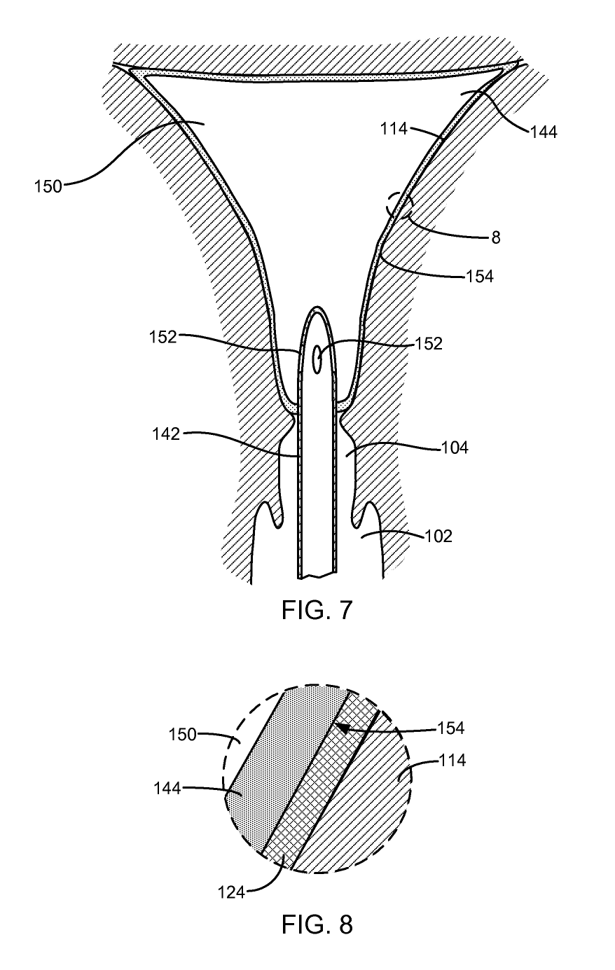

At 174, the coating of adjuvant 124 is applied to tissue of the uterine wall 114 or other tissue within the uterus 106 using the applicator 140. In accordance with one embodiment, the balloon 144 is expanded to the inflated state to drive the exterior surface 154, with the attached coating of adjuvant 124, against the uterine wall 100 of the patient, as illustrated in FIG. 7. The exterior surface 154 preferably conforms to the surfaces of the uterus to provide wide even coverage of the uterine wall 114 with adjuvant 124. The expansion of the balloon 144 also operates to spread the adjuvant 124 across the surface of the uterine wall and press the adjuvant 124 into crevices of the uterine wall. FIG. 8 is a magnified view of the region of FIG. 7 within circle 8 and illustrates the exterior surface 154 of the balloon 144 pressing the adjuvant 124 against the endometrium 116 of the uterine wall 114. In one embodiment, the balloon 144 is maintained in the expanded state (FIG. 7) for a predetermined amount of time to provide the desired application of the adjuvant 124 to the uterine wall 114 and to ensure absorption of the adjuvant into the endometrial layers 116.

In one embodiment, the adjuvant application 140 comprises a sealing portion 176, which is configured to engage the inner os of the cervix 104 and provide a seal that prevents the adjuvant 124 from entering the cervix 104. In one embodiment, the sealing portion comprises an inferior portion of the balloon 144 the provides the sealing function upon inflation of the balloon 144. In another embodiment, an elastomeric member 178 (FIG. 7) is attached to the tube 142 adjacent the balloon 144 and provides a seal to prevent the adjuvant from entering the cervix 104.

At 180, the balloon 144 is removed from the uterus of the patient. In one embodiment, the balloon 144 is returned to the deflated state (FIG. 6) prior to its removal from the uterus 106. This results in a layer of adjuvant 124 on the uterine wall 114, as illustrated in FIG. 2.

At 182, the adjuvant coated uterine wall 114 is exposed to laser light using, for example, the surgical system illustrated in FIG. 1. In one embodiment, this exposure of the adjuvant coated tissue causes the ablation of the tissue for the purpose of treating a condition of the patient. In one embodiment, the condition is menorrhagia. In other embodiment, the adjuvant coated tissue includes fibroids, the exposure of which to laser light ablates the fibroids.

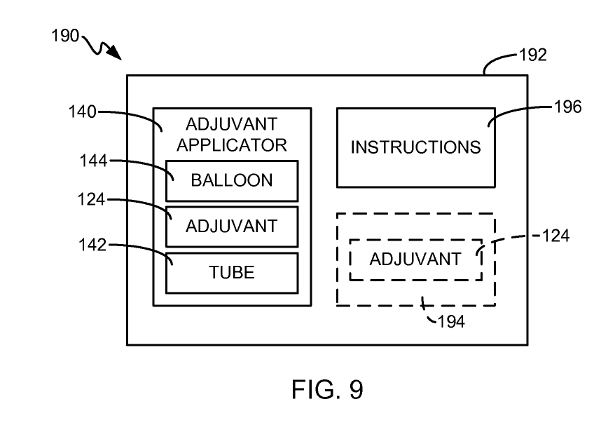

Another embodiment is directed to a kit 190, such as that illustrated in the block diagram of FIG. 9. One embodiment of the kit 190 comprises one or more of the embodiments of the adjuvant applicator 140 and the adjuvant 124 described herein. The kit 190 also includes a package 192 containing the adjuvant applicator 140 and the adjuvant 124. In one embodiment, the exterior surface 154 of the applicator 140 is coated with the adjuvant, such that it is prepared for insertion in the patient upon opening the package 192. In accordance with another embodiment, the balloon 144 is not coated with the adjuvant 124. Instead, the adjuvant 124 is stored in a suitable container 194 and is applied to the exterior surface 154 of the applicator 140 prior to its insertion into the patient. In one embodiment, the kit comprises instructions 196 for using the applicator 140.

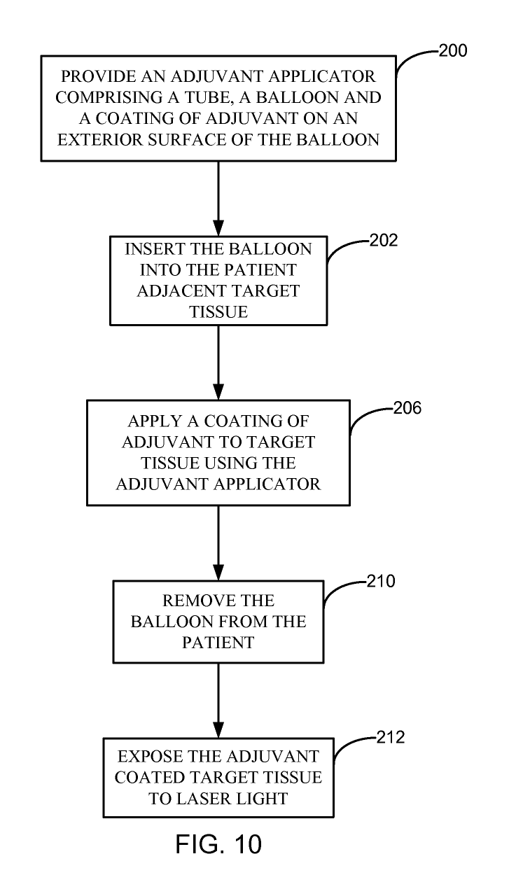

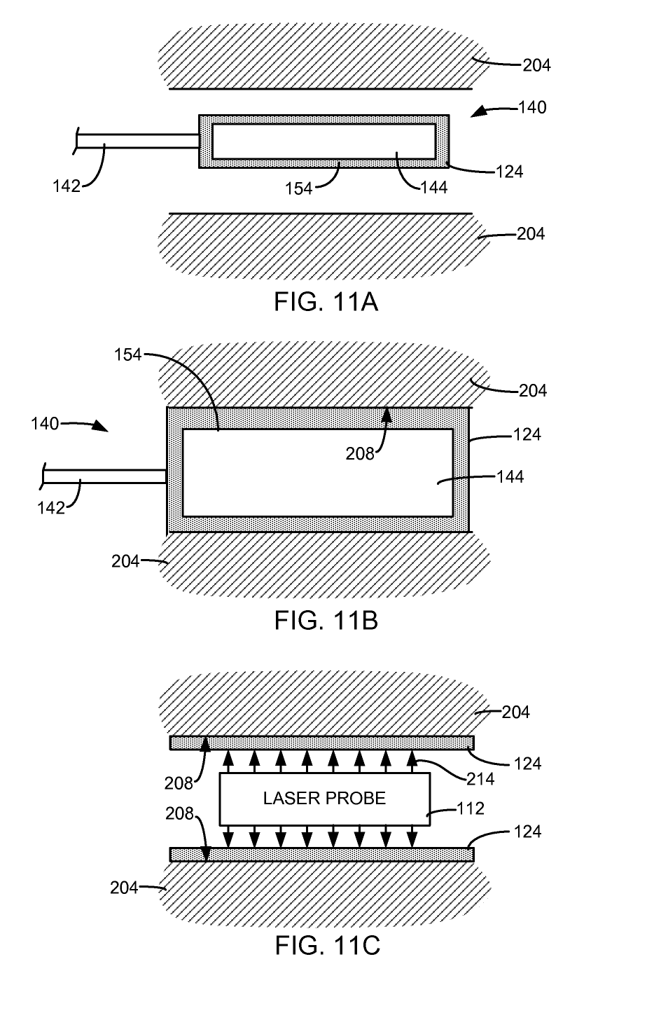

While embodiments of the invention have been described as being used to apply a coating of adjuvant to a uterine wall of a female patient for the purpose of enhancing laser ablation of the uterine wall, those skilled in the art understand that the disclosed adjuvant applicator 140 and method (FIG. 5) may also be used in the treatment of other tissues of female or male patients. FIGS. 10 and 11A-11C illustrate the application of embodiments of the present invention to other tissues of a patient.

FIG. 10 is a flowchart illustrating a method of applying an adjuvant to target tissue of a patient in accordance with embodiments of the invention. FIGS. 11A-11C are simplified drawings illustrating steps of the method of FIG. 10. At 200, an adjuvant applicator 140 comprising a tube 142, a balloon 144 and a coating of adjuvant on an exterior surface 154 of the balloon 144 is provided.

At 202, the balloon 144 is inserted into the patient adjacent target tissue 204, as illustrated in FIG. 11A. In one embodiment, the balloon 144 is in the deflated state when inserted into the patient adjacent the target tissue 204. The target tissue 204 can be any tissue that the balloon 144 may be placed adjacent to or that is capable of receiving the balloon 144 in the deflated state. In one embodiment, the target tissue is a tissue located in the pelvic region of the patient. Exemplary embodiments of the target tissue 204 include the uterus, the colon, the rectum, the prostate, the vagina, the cervix, the urethra, the bladder, the anus, the intestines or other pelvic tissue of the patient. Exemplary non-pelvic tissues include the esophagus, or tissue of the esophagus.

At 206, a coating of adjuvant 124 is applied to the target tissue 204 using the applicator 140. In one embodiment, the balloon 144 is expanded from the deflated state to the inflated state to drive the exterior surface 154, with the attached coating of adjuvant 124, against the wall 208 of the target tissue 204, as illustrated in FIG. 11B. The inflation of the balloon 144 also operates to spread the adjuvant 124 across the surface of the tissue 204 and press the adjuvant 124 into the wall 208.

At 210, the balloon 144 is removed from the patient. In one embodiment, the balloon 144 is deflated prior to its removal from adjacent the target tissue 204. The removal of the balloon 144 from adjacent the target tissue 204, leaves behind a coating of adjuvant 124 on the surface 208 of the target tissue 204, as illustrated in FIG. 11C.

At 212, the adjuvant coated target tissue 204 is exposed to laser light 214 using, for example, laser probe 112 of the surgical laser system 100 described above, to ablate the target tissue 204 or perform another laser procedure.

In accordance with another embodiment of the invention, the adjuvant applicator 140 is provided (step 200) and the balloon 144 is inserted in the patient adjacent the target tissue 204 (step 202; FIG. 11A). The balloon 144 is then filled with a hot or cold (e.g., cryogenic) gas or liquid to expand the balloon 144 to the inflated state (FIG. 11B). With the balloon in the expanded state, thermal energy is conducted through the balloon 144 to the adjuvant coated target tissue 204 to ablate the tissue 204. In accordance with one embodiment, the adjuvant 124 is selected to enhance thermal damage to the target tissue 204. Exemplary embodiments of the adjuvant include cytokines, chemotherapeutic agents, nanoparticles, or other suitable adjuvant. In one embodiment, the adjuvant 124 operates to enhance damage to the tissue 204 as though the thermal energy conducted from the balloon 144 is greater than that actually conducted. Thus, the ablation performed with the adjuvant 124 is more rapid than what would be possible using the same thermal energy without the adjuvant coating on the target tissue 204. As a result, ablation processes can be conducted more quickly. In accordance with this embodiment, the balloon 144 is deflated and removed from the patient following the ablation of the tissue 204. Accordingly, step 212 is not performed. The coating of adjuvant on the tissue 204 may be removed from the tissue 204 after the balloon 144 is removed.

Although the present invention has been described with reference to preferred embodiments, workers skilled in the art will recognize that changes may be made in form and detail without departing from the spirit and scope of the invention.

1-5. (canceled)

6. A method of treating a condition of a patient comprising:
providing an adjuvant applicator comprising:
tubing;
a balloon coupled to the distal end of the tubing and having an inflated state and a deflated state, which are reached through the passage of gas or liquid through the tubing; and
a coating of an adjuvant on an exterior surface of the balloon;
inserting the balloon in the deflated state in the patient adjacent target tissue of the patient;
applying the coating of adjuvant to the target tissue using the adjuvant applicator comprising:
inflating the balloon to an inflated state wherein the inflated state expands the exterior surface of the balloon such that the adjuvant contacts the target tissue; and
removing the balloon from the patient; and
exposing the adjuvant coated target tissue to the laser light;
ablating the target tissue responsive to exposing the adjuvant coated target tissue to the laser light.

7. The method of claim 6, wherein the adjuvant is suspended in a gel.

8. (canceled)

9. The method of claim 6, wherein applying a coating of adjuvant to the target tissue comprises maintaining the balloon in the inflated stated for a predetermined period of time prior to deflating the balloon, wherein a portion of the adjuvant remains adhered to the target tissue.

10. The method of claim 6, wherein applying a coating of adjuvant to target tissue comprises deflating the balloon prior to removing the balloon from the patient.

11. The method of claim 6, wherein exposing the adjuvant coated target tissue to the laser light comprises exposing the adjuvant coated target tissue to laser light having a wavelength of approximately 532 nm.

12. The method of claim 6, wherein the target tissue is selected from the group consisting of the uterus, the colon, the rectum, the prostate, the vagina, the cervix, the urethra, the bladder, the anus, the intestines and the esophagus.

13. The method of claim 6, wherein inserting the balloon in the deflated state in the patient adjacent target tissue, comprises inserting the balloon in the deflated state in the uterus of the patient, wherein the target tissue comprises the uterine wall.

14. The method of claim 13, wherein inflating the balloon to the inflated state creates a seal between the uterus and the cervix of the patient.

15. A kit comprising:
an adjuvant applicator comprising:
tubing; and
a balloon coupled to the distal end of the tubing and having an inflated state and a deflated state, which are reached through the passage of gas through the tubing;
an adjuvant; and
a package containing the adjuvant applicator and the adjuvant.

16. The kit of claim 15, further comprising instructions for using the adjuvant applicator.

17. The kit of claim 15, wherein the adjuvant is configured to absorb energy from laser light having a wavelength of approximately 532 nm.

18. A method of treating a condition of a female patient comprising:
providing an adjuvant applicator comprising:
tubing;
a balloon coupled to the distal end of the tubing and having an inflated state and a deflated state, which are reached through the passage of gas or liquid through the tubing; and
a coating of an adjuvant on an exterior surface of the balloon;
inserting the balloon in the deflated state in the uterus of the patient;
applying the coating of adjuvant to the uterine wall of the patient using the adjuvant applicator comprising:
inflating the balloon to an inflated state wherein the inflated state expands the exterior surface of the balloon such that the adjuvant contacts the uterine wall; and
removing the balloon from the patient; and
exposing the adjuvant coated uterine wall to laser light;
ablating tissue of the uterine wall responsive to exposing the adjuvant coated target tissue to the laser light.

19. The method of claim 18, wherein the tissue of the uterine wall comprises the endometrium of the uterine wall.

20. The method of claim 18, wherein the tissue of the uterine wall comprises fibroids.
